(19) United States
(12) Patent Application Publication
(10) Pub. No.: US 2021/0015514 A1
(43) Pub. Date: Jan. 21, 2021

(54) SURGICAL RETRACTOR TOOL

(71) Applicant: Norwegian University of Science and Technology, Trondheim (NO)

(72) Inventors: Per Even STORLI, Trondheim (NO); Lars Cato REKSTAD, Trondheim (NO); Brynjulf YSTGAARD, Trondheim (NO)

(21) Appl. No.: 16/623,027

(22) PCT Filed: Jun. 8, 2018
(86) PCT No.: PCT/EP2018/065240

§ 371 Date: Dec. 16, 2019

(30) Foreign Application Priority Data
Jun. 16, 2017 (GB) 1709595.1

(51) Int. Cl. A61B 17/29; A61B 17/28; A61B 17/22

(52) U.S. Cl. CPC A61B 17/29; A61B 17/282; A61B 17/22031; A61B 2017/00818; A61B 2017/2926; A61B 2017/22035; A61B 2017/292; A61B 2017/2825

(57) ABSTRACT

A surgical retractor tool for removing an object from the stomach or from the oesophagus. The tool includes a flexible steerable tube for insertion into the body via the oral cavity and a gripper at the distal end of the tube for gripping an object in the stomach or the oesophagus in order to allow for removal of the object from the stomach or the oesophagus. The gripper includes first and second rotatable gripper surfaces and a clamping mechanism for moving the gripper surfaces to clamp the object. The rotatable gripper surfaces are arranged to rotate relative to the tube in order to allow a longitudinal axis of a gripped object to move into alignment with a longitudinal axis of the tube.

The invention relates to a surgical retractor tool, in particular to a tool for removing objects from the oesophagus or from the stomach.

There are various circumstances in which a surgeon may wish to remove objects from the body and in particular from the oesophagus or from the stomach. For example, children commonly swallow small objects such as batteries, marbles, toys and so on either by accident or on purpose. Animals may also commonly swallow various foreign objects including the same objects that children may swallow, as well as other items such as stones, sticks, bones and so on. It is preferred to carry out procedures to remove such objects without the need for any surgical incision, for example by retrieving objects from the stomach using a tool that is inserted through the oral cavity and guided by an endoscope, rather than by using a laparotomy. In most cases the objects that are in the stomach are relatively small objects that have been easily ingested, and may have been swallowed by mistake. This allows for relatively straightforward endoscopical removal of the objects using conventional surgical grasping tools or wire nets/loops for hooking on the object.

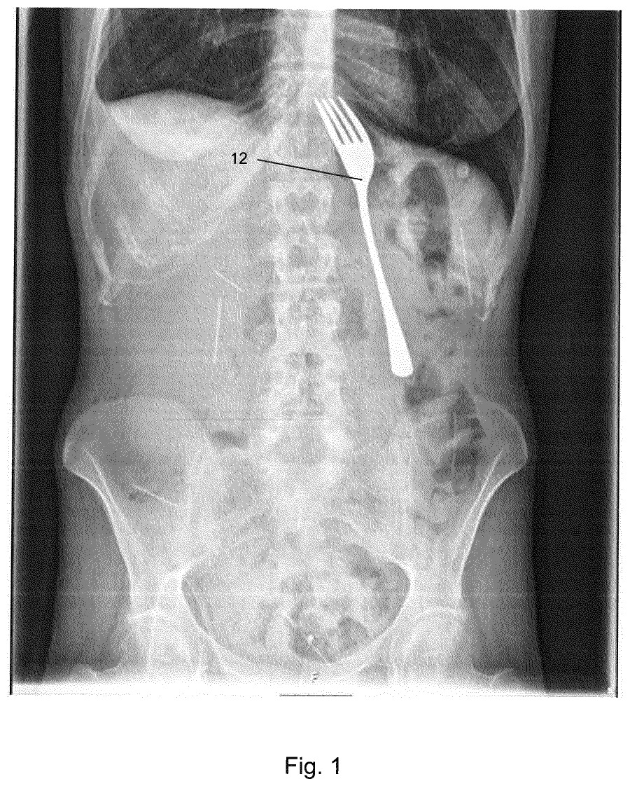

A more difficult situation arises when relatively large and/or sharp objects are swallowed. Some patients deliberately swallow relatively long objects such as cutlery as shown in the X-ray image of FIG. 1. This can be due to mental illness, as a result of a patient attempting to gain access to drugs and/or due to a patient attempting to be placed in a hospital for other reasons. Some psychiatric patients compulsively swallow various types of objects. It will be seen from a close inspection of FIG. 1 that the stomach and digestive system also contains a number of metal pins and needles. Drug addicts may swallow large objects as a way to obtain treatment with strong painkillers. Imprisoned criminals have been known to swallow large objects in order to be moved from the prison to a hospital, which may be as part of an escape attempt. Children and animals might also accidentally or deliberately swallow large objects.

It is difficult for large and/or sharp objects such as cutlery to be removed using surgical tools inserted by mouth. This can be a difficult and time consuming procedure using up valuable hospital resources. In many cases it is not possible to retrieve the object via the oesophagus, or the risk of causing harm hinders such an attempt and therefore a more invasive surgical procedure is needed, such as removal of the object by laparotomy. This will consume even more time and resources than an endoscopic procedure. Often the patients that swallow such objects will do so more than once and thus become ‘repeat customers’ of their health service. Most hospitals will need to devote considerable time and resources to such patients and this may be to the detriment of the treatment of other patients. Animals are known to swallow large objects as well, which creates similar problems for veterinary surgeons. An improved system for retrieving such objects endoscopically would be beneficial both for the hospital and the patients treated. The inventors have developed a device for a more straightforward removal of objects from the stomach, including large objects such as cutlery.

Viewed from a first aspect, the invention provides a surgical retractor tool for removing an object from the oesophagus or from the stomach, the tool comprising: a flexible steerable tube for insertion into the body via the oral cavity; and a gripper at the distal end of the tube for gripping an object in the oesophagus or the stomach in order to allow for removal of the object from the oesophagus or from the stomach; wherein the gripper includes: a first gripper surface, a second gripper surface, and a clamping mechanism for moving the gripper surfaces relative to one another to thereby clamp the object between the two gripper surfaces, wherein the two gripper surfaces are rotatable gripper surfaces that are arranged to rotate relative to the tube in order to allow a longitudinal axis of a gripped object to move into alignment with a longitudinal axis of the tube.

With this tool it becomes possible to remove relatively long objects from the oesophagus or the stomach, such as cutlery and other objects of a similar size. Whilst there are numerous tools that may be used to remove smaller objects from the oesophagus or the stomach (such as coins or small batteries) the inventors have found that so far there is no effective solution for removal of longer objects such as cutlery, which may have a length of over 15 cm, such as a length of about 20 cm or more, sharp objects such as razor blade or both such as a steak knife. The retractor tool of the first aspect can remove such objects. These sharp and/or longer objects ideally need to be gripped at one end. Typically the ends of the object will be close to the oesophagus or stomach wall or may even be pushed into the oesophagus or stomach wall. As well as this, longer objects also generally require re-orientation before they can safely be removed from the stomach. By the use of a flexible tube having a gripper at the distal end with rotatable gripper surfaces then it becomes possible to more easily access an end of a long object, as well as to allow the object to be drawn out of the stomach with the object's longitudinal axis aligned with the axis of the tube. When the rotating surfaces grip the object at its end and then tension is applied to the tube to remove it from the body, then the gripper surfaces will rotate to allow the object to be removed from the stomach smoothly. The length of the object will be brought into alignment with the longitudinal axis of the tube while the tube passes back through the oesophagus and oral cavity.

It is preferred for the gripper surfaces to resiliently grip the object. For example the gripper surfaces may be provided by a layer of an elastic material. It is also preferred for the gripper surface to be able to generate a relatively high degree of friction when in contact with a metal object. For example, the gripper surface should have higher friction in contact with a metal object than a metal-metal contact. One intended use for the tool is to remove cutlery from the stomach or the oesophagus and this is traditionally done using stainless steel surgical implements as discussed above. Advantageously the gripper surfaces may comprise a material that is able to provide increased friction in contact with metal cutlery, such as stainless steel cutlery, when compared to the friction between two stainless steel surfaces, such as between stainless steel cutlery and known surgical tools made of stainless steel. The level of friction may be similar to that provided by a rubber-metal contact and the gripper surface may be provided by a rubber material such as a silicone rubber or latex. It should be noted that it is not essential for the gripper to have a soft or resilient form as provided by rubber, but instead the level of friction is more important. The same level of friction can be provided by hard surfaces having an appropriate gripping surface, such as a roughed surface or a sandpaper-like surface with particles of a hard material included to increase friction.

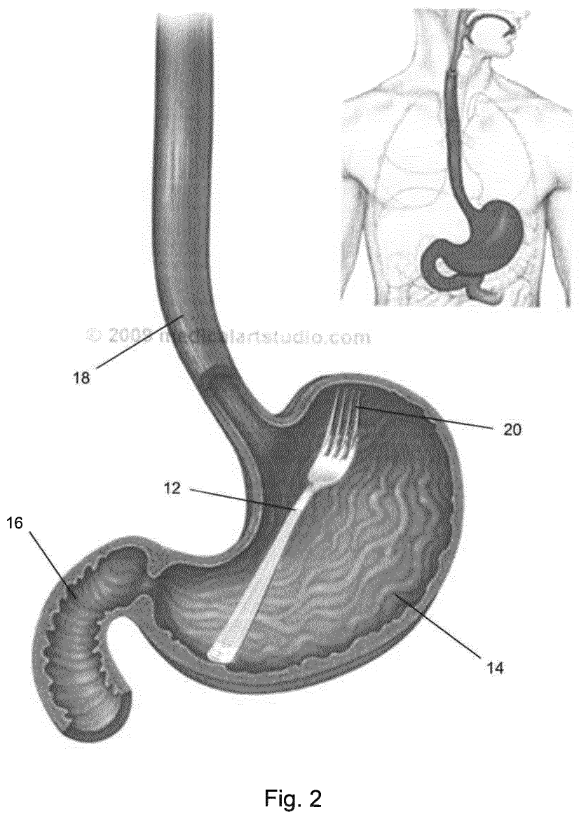

The gripper may advantageously be joined to the tube at an articulated end of the tube in order that the gripper can be steered and directed toward the object, such as toward an end of the object, by use of the same mechanism that steers and directs the tube. In this way the gripper can be directed toward an end of the object in order to grip the end of the object no matter where the object sits in the stomach. For example, the gripper can be directed at various angles after entering the stomach from the oesophagus, including at an angle greater than 90 degrees to the longitudinal axis of the endoscope so that the gripper curves back toward the oesophagus. This means that the end of a long object can be gripped even when it is located around a corner from the end of the oesophagus, such as the tines of the fork in the situation shown in FIG. 2. Suitable mechanisms for steering the flexible tube are known, and the known mechanisms include various ways of actuating the steerable end of the tube to direct it in a required orientation.

The steerable flexible tube may be a steerable endoscope. This allows for the surgeon to both view the object and grip the object using a single tool. Thus, a retractor tool with an endoscope incorporated as the steerable tube may not need any other imaging system to be used to allow the surgeon to retrieve the object. As is known in the art, an endoscope allows the surgeon to view inside the body, with the viewing angle being typically directed along the axis of the endoscope at the distal end. Thus, as the endoscope is steered then the viewing angle is also steered. With the proposed retractor tool the viewing angle of the endoscope may advantageously be directed along an axis of the gripper and between the two gripper surfaces when the gripper is open. The surgeon can accurately place the gripper in relation to the object, since they will easily see when the end of the object is in between the gripper surfaces.

The gripper surfaces are mounted for rotation relative to the remainder of the gripper. When the gripper is closed then the axis of rotation of the gripper surfaces may be perpendicular to a longitudinal axis of the tube, or within 10 degrees of perpendicular. It will be understood that in the case where the tube is curved at the articulated end then the longitudinal axis follows the curve, and the perpendicular may be a perpendicular to a tangent of this curve where the gripper is located. Preferably the gripper surfaces can be allowed to rotate freely in order that the object can assume a natural position with minimum resistance as it is removed from the stomach. The gripper surfaces may be arranged to always freely rotate, or optionally there may be a possibility for controlled rotation of at least one of the gripper surfaces, for example rotation driven by a rotating actuator. This could be useful to prompt a required movement of the object in some situations.

The rotation of the gripper surfaces may be a rotational movement around a centre of the gripper surfaces. The gripper surfaces may be circular or elliptical. For example, they each gripper surface may have a disc shape. Using rotational symmetry in this way means that there is no need to have the gripper surfaces in any particular orientation. It also ensures that they can easily rotate to align an object with the longitudinal axis of the tube.

In order for safe removal of a range of objects including objects with sharp points or edges then the gripper surfaces should have a surface area and shape sufficient to enclose the expected sharp points or edges. The gripper surfaces may be shaped and sized in order that they can enclose the tines of a fork. Thus, the width of the gripper surfaces may be at least as large as the width of the tines of a fork, which might typically have a width of 1.8-2.2 cm. Thus, in some examples the gripper surfaces have a width of at least 2 cm, or at least 3 cm. In the case of a circular gripper surface the width is the diameter. The retractor tool may be provided with removable and replaceable gripper surfaces of different sizes. This allows for sufficiently large gripper surfaces to be used to grip the object of concern, whilst avoiding the need to pass an unnecessarily wide gripper surface through the oesophagus.

Advantageously the rotation of the gripper surfaces is a rotation about an axis that extends outwardly from the gripper surface. For example, the gripper surfaces may be arranged to rotate about an axis that is normal to the gripper surface, or within 10 degrees of normal. Typically the gripper surfaces may be flat surfaces and thus they may be arranged to rotate within a plane of the surface. This allows for unimpeded rotation when the gripper surfaces are facing each other in the same orientation. However, the gripper surfaces may also be mounted so that they may tilt relative to the axis of rotation, for example they may tilt by up to 10 degrees or up to 20 degrees relative to alignment with the plane of rotation. A ball and socket connector may be used to allow for both rotation and tilting. The ability for the gripper surface to tilt can enable a better grip when the object to be gripped is not a flat shape, such as the curved tines of a fork or the end of a spoon. The tilting may be arranged to occur freely such that the gripper surfaces can adopt the most natural position to apply force to the object when the gripper mechanism urges the gripper surfaces toward one another.

Each of the gripper surfaces may be a surface on a rotatable substrate. For example they may be surfaces of rotatable plates. The rotatable substrates may each be rotatably connected to a jaw of a pair of gripper jaws that are used to move the gripper surfaces toward and away from one another due to the action of the gripper mechanism.

The gripper surfaces and/or the rotatable substrates, where present, may have outer edges with features designed to minimise damage to the body as the tool is inserted or removed. Thus, the outer edges may have a curved shape and/or may be formed from a relatively soft or resilient material, such as a rubber material.

The clamping mechanism may use any suitable arrangement to move the gripper surfaces. This may be based on actuation using mechanical, hydraulic or electrical devices, for example. The clamping mechanism may use levers to move the gripper surfaces, such as hinged levers or levers connected together with a pivot mechanism. Such a clamping mechanism may be actuated using forces transmitted from a controller at a proximal end of the tube, i.e. outside of the body.

The gripper surfaces rotate to align the length of the removed object with the longitudinal axis of the tube and this is hence a rotation about a first axis of rotation that extends perpendicular to the longitudinal axis. In addition to this rotation about the first axis the retractor tool may also be arranged for a rotation of the gripper surfaces about a second axis that is orthogonal to the first axis. This may be a pivoting movement of the gripper tool relative to the distal end or a twisting rotation of the gripper surfaces about the longitudinal axis of the tube. Both of the pivoting and twisting movement may be used, thus allowing for rotation about the longitudinal axis of the tube as well as about two orthogonal axes. Having additional degrees of freedom for rotation can be useful in order to orient the gripper surfaces to align them with the object to be removed. Whilst the steerable tube may be twisted at the proximal end in order to twist the distal end of the tube, this may not easily provide a suitably aligned orientation of the gripper surfaces in situations where the articulated end of the tube needs to be used to direct the gripper tool around a corner to thereby grasp an end of the object. The retractor tool may optionally be arranged to allow for longitudinal movement of the gripper surfaces relative to the distal end of the tube. This can allow for extra ‘reach’ in situations where further movement of the tube into or out of the body is not easy to achieve or might move the gripper surfaces out of alignment with a target area on the object.

The retractor tool may be provided with a protective sheath for preventing the object from damaging the body as it is removed from the body. For example there may be a sheath of flexible material, such as a rubber sheath, that fits along a body of the tube as the retractor tool is inserted into the body and that slides or rolls in order to cover the gripper as the retractor tool is removed from the stomach. The sheath may be arranged to fold over the gripper via an eversion fold that moves downward along the tube as the retractor tool is pulled upward along the oesophagus and removed from the stomach.

As the retractor tool is for use within the body then the materials of the retractor tool should be safe for surgical use. For example, the gripper surfaces may be silicone rubber and the other parts of the gripper may be stainless steel. The gripper may have exchangeable gripper surfaces as set out above, which may be provided along with relevant other parts such as a supporting plate or similar. The gripper itself may be removable so that the steerable tube can be used for other purposes. This has particular advantages where the tube is an endoscope, since flexible endoscopes can be complicated and hence expensive leading to benefits if they can be used in multiple different procedures. In relation to the connection of the gripper to the end of the tube this may use a re-usable mechanical coupling such as a screw fit or bayonet coupling, or alternatively it may use single use parts such as adhesive tape and/or a shrink wrap.

A gripper tool head that can be mounted to a tube to form a retractor tool is considered to be novel and inventive in its own right. Therefore, viewed from a second aspect, the invention provides a gripper tool head arranged for connection to a distal end of a flexible steerable tube, such as an endoscope, in order to form the retractor tool of the first aspect. Thus, the gripper tool head may be arranged to be coupled to the end of the steerable tube and to a suitable actuation device for the gripper mechanism such as a rod or wire arrangement. As with the gripper discussed above the connection to the distal end of the steerable tube may use a re-usable mechanical coupling such as a screw fit or bayonet coupling, or alternatively it may use single use parts such as adhesive tape and/or a shrink wrap. With the gripper tool head of the second aspect it becomes possible to adapt an existing steerable tube to make the retractor tool of the first aspect. This can be beneficial since, as noted above, a steerable tube such as an endoscope may be expensive and it can hence be desirable to allow for the gripper to be fitted to an existing system. The gripper tool head includes a gripper as in the first aspect and the gripper may include other features as discussed above.

The invention extends to the use of the retractor tool for removal of an object from the oesophagus or from the stomach. Thus, in a further aspect the invention provides a method for removal of an object from the oesophagus or the stomach using a retractor tool as in the first aspect, the method comprising: inserting the gripper into the oesophagus and optionally into the stomach via the oral cavity; using the gripper surfaces to clamp the object; and withdrawing the tube and gripper along with the object from the oesophagus or the stomach whilst allowing the gripper surfaces to rotate to thereby align the longitudinal axis of the object with the longitudinal axis of the tube. The method may include using a retractor tool with an endoscope as the steerable tube, wherein the endoscope is used to navigate the gripper to the correct position relative to the object. The object may be clamped at one end thereof. In the case of cutlery the object may be clamped at the non-handle end, i.e. the tines of a fork or the blade of a knife. The method may include using a retractor tool with any of the other features set out above.

An example embodiment of the invention will now be described by way of example only and with reference to the accompanying drawings, in which:

FIG. 1 shows an X-ray of a patient that has swallowed various foreign objects;

FIG. 2 illustrates a typical placement of a fork in the stomach after it has been swallowed;

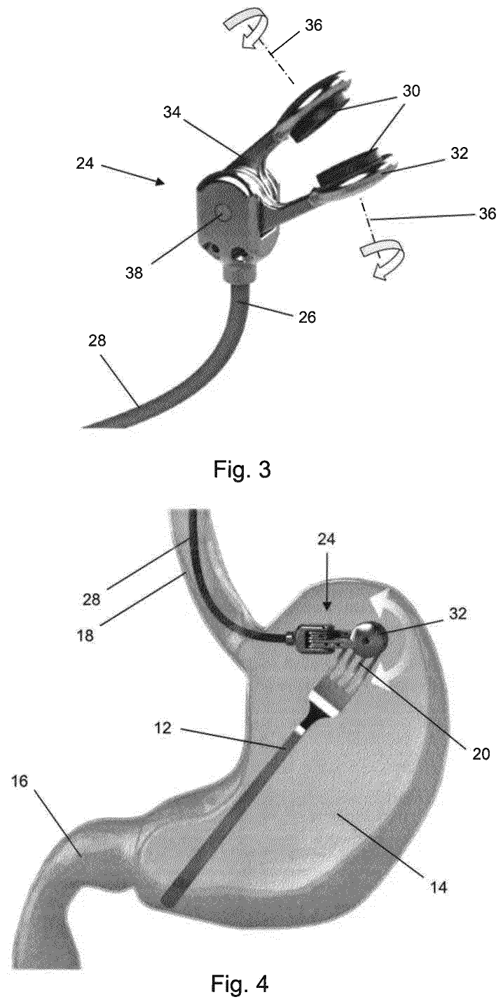

FIG. 3 shows a surgical retractor tool;

FIG. 4 shows the tool of FIG. 3 being directed toward the upper end of a fork in the stomach;

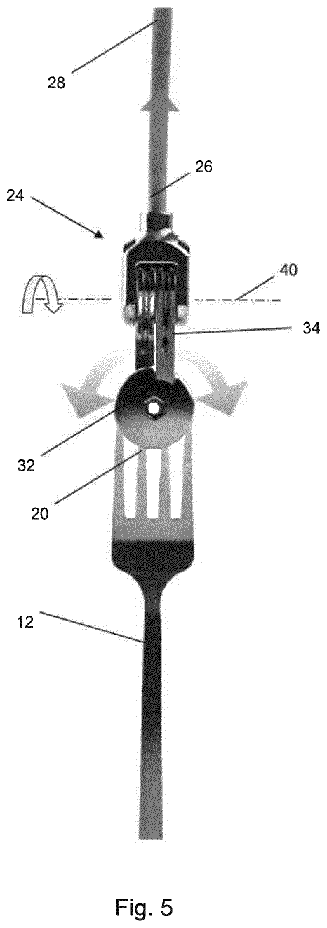

FIG. 5 shows a fork gripped by the tool of FIG. 3 and being aligned with the longitudinal axis of a steerable tube of the tool;

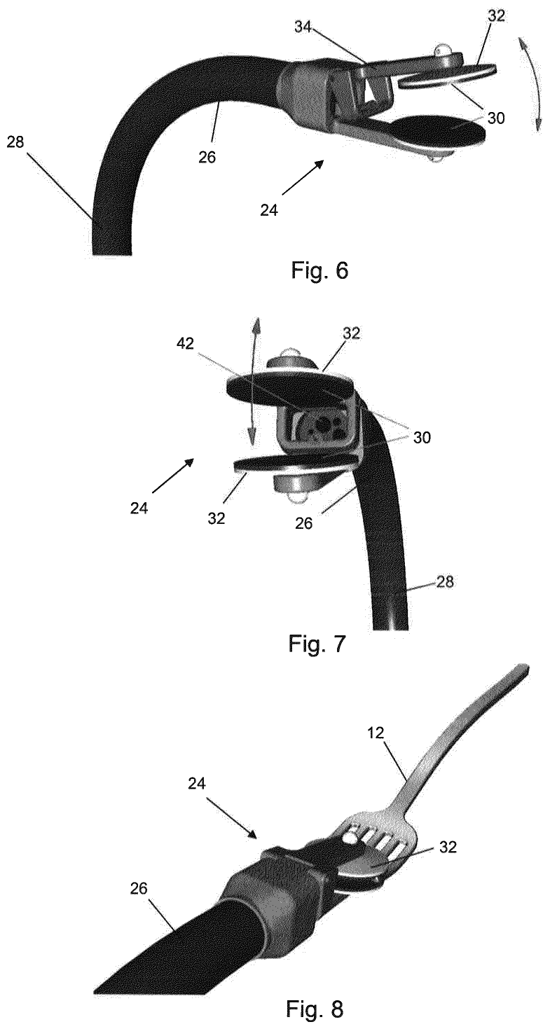

FIG. 6 shows another surgical retractor tool;

FIG. 7 shows the tool of FIG. 6 in a different orientation; and

FIG. 8 shows the tool of FIG. 6 gripping a fork.

FIG. 1 and FIG. 2 illustrate a challenging situation for a surgeon where a patient has deliberately swallowed a fork. A large object such as a fork 12 will become lodged in the stomach 14 when it is swallowed, since it is not possible for it to readily reorient itself in order to leave the stomach 14 via the duodenum 16 or the oesophagus 18. When a long object 12 enters the stomach 14 from the oesophagus 18 then one end of the object 12 will rest at the antrum (the base) of the stomach 14. The other end of the object 12 will be passed out of the oesophagus 18 and then come to rest in contact with the opposite wall of the stomach 14, i.e. at the fundus (the top) of the stomach 14 with the patient in the standing orientation as shown for the fork 12 in FIGS. 1 and 2.

It will be appreciated that challenges arise in removing such an object 12. It is necessary to grip an end of the object 12, ideally the upper end 20, and this is hard to achieve with a tool that enters the stomach 14 via the oesophagus 18. If the object 12 is gripped at the middle then it may be physically impossible to remove it from the stomach 14, and the ends of the object can cause injury to the patient. As well as this, a strong grip may be needed in order to pull the object 12 into a position where the upper end 20 can be directed into the oesophagus 18 since this can require distortion of the stomach 14 as well as movement of the object within the stomach 14. Conventional surgical grasping tools often have relatively flexible metal grasping ends and these will not easily give a suitable grip strength and manoeuvrability.

A surgical retractor tool for removal of objects from the stomach 14 is shown in FIGS. 3, 4 and 5. The tool includes a gripper 24 mounted at a distal end 26 of a flexible steerable tube 28 that has an articulated/steerable end section. The features of such a steerable tube 28 are known and will not be described in detail. Any suitable mechanism may be used to steer the end of the device as the retractor tool is being inserted into the body. The steerable tube 28 may be an endoscope and thus may also include a camera or other means for allowing the surgeon to view the inside of the body. An example of this type is shown in FIGS. 6 to 8, as discussed below. Where an endoscope is used as the steerable tube 28 then the camera is advantageously arranged to look along the axis of the gripper 24 so that the location of the gripper 24 relative to an object 12 in the stomach 14 can easily be determined.

FIG. 3 shows the distal end 26 of the retractor tool with the steerable tube 28 curved at an angle, the gripper 24 open, and the gripper 24 pivoted relative to the axis of the steerable tube 28. The gripper 24 has first and second gripper surfaces 30 which are each located on rotatable discs 32 on two jaws of a pincer mechanism 34. The pincer mechanism 34 forms a clamping mechanism 34 for moving the gripper surfaces 30 relative to one another to thereby clamp the object 12 between the two gripper surfaces 30. Since the gripper surfaces 30 are mounted on the rotatable discs 32 then when the object 12 has been gripped it can rotate relative to the steerable tube 28. This allows the retractor tool to first grip the object 12 at one end 20 as shown in FIG. 4, and to then pull the end 20 of the object 12 into the oesophagus 18 whilst simultaneously bringing a longitudinal axis of the object 12 into alignment with a longitudinal axis of the tube 28. This alignment is shown in FIG. 5.

The pincer mechanism 34 can be actuated by a wire, which in the illustrated embodiment runs along the inside of the steerable tube 28 (or endoscope 28). Applying tension to the wire may urge the pincer mechanism 34 to a closed position and hence may apply a force to draw the gripper surfaces 30 together and to grasp an object between them. The wire may be able to be locked in place to keep an object 12 gripped between the gripper surfaces 30 of the gripper 24. Elasticity in the wire along with tension forces whilst the wire is locked in place can allow for a sustained gripping force on the object 12. A spring may be incorporated in the pincer mechanism 34 to open the gripper 24 when tension in the wire is released. In an alternative arrangement (not shown) the wire may run outside of the steerable tube 28, and for example may pass through a sheath extending along an outer surface of the steerable tube 28.

When the jaws of the gripper 24 are closed then the gripper surfaces 30 and their associated rotatable discs 32 can freely rotate about an axis of rotation 36 that is perpendicular to the longitudinal axis of the steerable tube 28 and passes through the centres of the gripper surfaces 30. The two jaws of the pincer mechanism 34 can also be rotated around a shaft 38 with an axis of rotation 40 that is perpendicular to the longitudinal axis of the steerable tube 28 and orthogonal to the gripper surface axis of rotation 36. In addition, the gripper 24 may optionally be able to twist around the longitudinal axis of the steerable tube 28. This can add a further degree of freedom when trying to place the gripper 24 at the end 20 of the object 12 in the stomach 14.

As noted above, FIGS. 6 to 8 show an example of a retractor tool where the steerable tube 28 is an endoscope. The endoscope 28 includes a camera 50 or other means for allowing the surgeon to view the inside of the body. FIG. 7 is a view looking back along the viewing angle of the endoscope 28 so that parts of the camera 50 can be seen. As is evident from FIGS. 6 and 7 the camera 50 is arranged to look along the axis of the gripper 24 so that the location of the gripper 24 relative to an object 12 in the stomach 14 can easily be determined. The gripper 24 is adapted in order to allow for an opening along the viewing angle of the camera 50. Other features of this example are generally similar to those explained above, and hence the retractor tool has first and second gripper surfaces 30 which are each located on rotatable discs 32 on two jaws of a pincer mechanism 34. In the same way as the retractor tool 22 of FIGS. 3 to 5 the pincer mechanism 34 forms a clamping mechanism 34 for moving the gripper surfaces 30 relative to one another to thereby clamp the object 12 between the two gripper surfaces 30, as shown with a fork 12 in FIG. 8. When gripped then the rotatable discs 32 allow for rotation of the gripper surfaces 30 so that the object 12 can rotate to come into alignment with the length of the endoscope 28.

We claim:

1. A surgical retractor tool for removing an object from the oesophagus or from the stomach, the tool comprising:
a flexible steerable tube for insertion into the body via the oral cavity; and
a gripper at the distal end of the tube for gripping an object in the oesophagus or the stomach in order to allow for removal of the object from the oesophagus or the stomach;
wherein the gripper includes:
a first rotatable gripper surface,
a second rotatable gripper surface, and
a clamping mechanism for moving the gripper surfaces relative to one another to thereby clamp the object between the two gripper surfaces,
wherein the rotatable gripper surfaces are arranged to rotate relative to the tube in order to allow a longitudinal axis of a gripped object to move into alignment with a longitudinal axis of the tube.

2. A surgical retractor tool as claimed in claim 1, wherein the gripper surfaces are provided by a layer of an elastic material that will generate higher friction in contact with a metal object than a metal-metal contact.

3. A surgical retractor tool as claimed in claim 2, wherein the level of friction is similar to that provided by a rubber-metal contact.

4. A surgical retractor tool as claimed in claim 1, wherein the gripper is joined to the steerable tube at an articulated end of the tube in order that the gripper can be steered and directed toward an end of the object by use of the same mechanism that steers and directs the tube.

5. A surgical retractor tool as claimed in claim 1, comprising an endoscope as the flexible steerable tube, wherein the viewing angle of the endoscope is along an axis of the gripper and between the two gripper surfaces.

6. A surgical retractor tool as claimed in claim 1, wherein the axis of rotation of the gripper surfaces, when the gripper is closed, is within 10 degrees of the perpendicular to the longitudinal axis of the tube.

7. A surgical retractor tool as claimed in claim 1, wherein the gripper surfaces are arranged to be able to rotate freely.

8. A surgical retractor tool as claimed in claim 1, wherein the gripper is arranged in order to allow for controlled rotation of at least one of the gripper surfaces as well as a free-wheeling rotation when desired.

9. A surgical retractor tool as claimed in claim 1, wherein the gripper surfaces are circular and the rotation of the gripper surfaces is a rotational movement around a centre of the circle.

10. A surgical retractor tool as claimed in claim 1, wherein the gripper surfaces are shaped and sized in order that they can enclose the tines of a fork.

11. A surgical retractor tool as claimed in claim 1, wherein the gripper surfaces have a width of at least 2 cm.

12. A surgical retractor tool as claimed in claim 1, wherein the gripper surfaces are removable and replaceable and the gripper is provided with additional removable and replaceable gripper surfaces of different sizes.

13. A surgical retractor tool as claimed in claim 1, wherein the rotation of the gripper surfaces is a rotation about an axis that extends outwardly from the gripper surface and within 10 degrees of a normal to the gripper surface.

14. A surgical retractor tool as claimed in claim 1, wherein the gripper surfaces are flat surfaces.

15. A surgical retractor tool as claimed in claim 1, wherein the gripper surfaces are mounted so that they can tilt relative to the axis of rotation.

16. A surgical retractor tool as claimed in claim 1, wherein the gripper surfaces are each mounted via a ball and socket connector.

17. A surgical retractor tool as claimed in claim 1, wherein in addition to the rotation of the gripper surfaces the retractor tool is arranged for a pivoting movement of the gripper relative to the tube and/or a twisting rotation of the gripper surfaces about the longitudinal axis of the tube.

18. A gripper tool head arranged for connection to a distal end of a steerable flexible tube in order to form a surgical retractor tool as claimed in claim 1.

19. A method for removal of an object from the stomach or from the oesophagus using a retractor tool as claimed in claim 1, the method comprising:
inserting the gripper into the oesophagus and optionally stomach via the oral cavity;
using the gripper surfaces to clamp the object; and
withdrawing the steerable tube and gripper along with the object from the stomach or the oesophagus whilst allowing the gripper surfaces to rotate to thereby align the longitudinal axis of the object with the longitudinal axis of the tube.
